(19) United States
(12) Patent Application Publication
(10) Pub. No.: US 2024/0069015 A1
(43) Pub. Date: Feb. 29, 2024

(54) DEVICE AND SYSTEM FOR THE DETECTION OF ENVIRONMENTAL CONTAMINANTS

(71) Applicants: UNIVERSITA' DEGLI STUDI DI BARI ALDO MORO (60%), Bari BA (IT); ITEL TELECOMUNICAZIONI S.R.L. (40%), Ruvo di Puglia (BA) (IT)

(72) Inventors: Antonio SCILIMATI, Bari BA (IT); Maria Grazia PERRONE, Bari BA (IT); Antonio GNONI, Bari BA (IT); Salvatore Maria SCACCO, Bari BA (IT); Michele DIAFERIA, Ruvo di Puglia (BA) (IT); Vincenzo DIMICCOLI, Ruvo di Puglia (BA) (IT); Anna TOLOMEO, Ruvo di Puglia (BA) (IT); Antonio RIGLIETTI, Ruvo di Puglia (BA) (IT); Davide SCILIMATI, Ruvo di Puglia (BA) (IT)

(21) Appl. No.: 18/259,841

(22) PCT Filed: Jan. 4, 2022
(86) PCT No.: PCT/IB2022/050040

§ 371 Date: Jun. 29, 2023

(30) Foreign Application Priority Data
Jan. 5, 2021 (IT) 102021000000134

(51) Int. Cl. G01N 33/543; G01N 33/569

(52) U.S. Cl. CPC G01N 33/54387; G01N 33/56983; G01N 2333/165

(57) ABSTRACT

The present invention relates to a device for identifying and monitoring possible environmental contamination by pathogenic and non-pathogenic microorganisms, e.g., viruses. The present invention further relates to a system comprising said device.

### STATE OF THE ART

The growing threat to global health security posed by the infection by SARS-CoV-2 (Severe Acute Respiratory Syndrome—Coronavirus-2), also referred to as the new 2019 coronavirus (2019-nCoV, or also 2019 nCoV-ARD), demonstrates how the globalization can exponentially accelerate the pandemic phenomena.

Coronavirus-2 is a viral strain of the SARS-related coronavirus/SARS-CoV species, part of the genus Betacoronavirus (Coronaviridae family), subgenus Sarbecovirus, discovered in late 2019. The official name given by the World Health Organization to the syndrome caused by said virus is Coronavirus disease-2019 (COVID-19). The SARS-CoV-2 virus results in a severe acute respiratory syndrome that can even lead to death. In particular, said SARS-CoV-2 virus is transmitted by the aerosols and, according to what is known so far, it takes about 66 minutes for the number of viable virus particles in said aerosols to be halved. Said virus maintains its viral load in the aerosols for hours, while on the surfaces even for days. 25% of the viral particles still retain the virulence after a little more than one hour, and after about 3 hours 12.5% of the viral load persists.

It has been demonstrated that SARS-CoV-2 can survive and possibly thrive in the environment, particularly on a variety of surfaces, such as printing and tissue paper, wood and fabric, outer layer of a surgical mask and on various clinical specimens. Overall, SARS-CoV-2 can be highly stable for quite some time in a favorable environment.

For example, on stainless steel, it takes 5 hours and 38 minutes to halve the viral load. On plastic, however, the half-life is 6 hours and 49 minutes.

In the 2003 SARS epidemic, the transmission associated with the wastewater aerosols was documented. As a result, there is a need to monitor the efficiency of the wastewater treatment systems and the possibility of transmission of the contamination by SARS-CoV-2 through the sewage systems.

The transmission of SARS-CoV-2 through aerosols and its permanence on the surfaces demonstrate the need to monitor both the air, particularly in closed environments, as well as water. In other words, it is necessary to be able to detect in the air, in particular of closed environments as well as in water, the presence of viruses and in general of microorganisms.

The coronavirus-2 pandemic, therefore, has increased the urgency of a long-standing need i.e., to detect contaminants in the air and water, particularly pathogenic and non-pathogenic microorganisms. Therefore, there is an increasing need to provide and develop devices and/or systems capable of detecting the presence of pathogenic microorganisms, such as for example viruses, in the air we breathe and in water for civil use. There is also a need for devices or systems that, in addition to being able to detect the presence of said pathogenic and non-pathogenic microorganisms, are able to quantify the concentration of said microorganisms that may possibly be present in the air or water.

### OBJECTS OF THE INVENTION

Object of the present invention is to provide a device which allows detecting environmental contaminants, such as microorganisms, e.g., pathogenic microorganisms, that may possibly be present in the air or water.

Another object of the present invention is to provide a device which allows detecting the concentration of said microorganisms present in the air or water.

Still an object of the present invention is to provide a device capable of detecting the presence of viruses, particularly the presence of the new SARS-CoV-2 coronavirus, in the air or water and determining its concentration.

Finally, an object of the present invention is to provide a system capable of detecting the presence of microorganisms, preferably viruses, even more preferably new SARS-CoV-2 coronavirus-2, in closed environments provided with air circulation and/or recirculation plants or systems, such as air conditioning plants or systems.

### DESCRIPTION OF THE INVENTION

Object of the invention is a device comprising a body, at least one hole, at least one filter, and at least one support, said at least one support being provided with a first part provided with at least one examination portion and with a second part provided with at least one control portion. Said support is placed inside the body of the device.

Said device has proven particularly effective in detecting environmental contaminants, such as pathogenic microorganisms like viruses, in the air or water. In particular, the device of the invention is capable of detecting the presence in the air and water of the new SARS-CoV-2 coronavirus.

This is especially beneficial for a virus that is transmitted through aerosols. In fact, the transmission of microorganisms, such as for example viruses, by aerosol obviously implies the presence of said microorganisms in the air, which is particularly dangerous when the microorganism present in the air is a pathogenic and particularly infectious microorganism such as SARS-CoV-2. In the present invention, said virus responsible for the syndrome referred to as COVID-19, is called SARS-CoV-2 (Severe Acute Respiratory Syndrome—Coronavirus-2), also referred to as new 2019 coronavirus (2019-nCoV, or also 2019 nCoV-ARD). Therefore, the abbreviation “SARS-CoV-2” refers to the new coronavirus of the Coronaviridae family, identified in late 2019, while the abbreviation “COVID-19” refers to the syndrome caused by said SARS-CoV-2 virus.

The device of the invention allows detecting the presence of microorganisms present in the air or water, since it comprises at least one support inserted within the body of said device, preferably constituted by a nitrocellulose membrane, on which antibodies capable of recognizing and binding a specific portion, called epitope, of said microorganism that is intended to be detected are adsorbed.

The epitope is the portion of an antigen that is recognized and bound by a given antibody. In other words, the antibody specific for a certain antigen binds said antigen in the epitope, which is precisely a portion of said antigen. The single antigen molecule may contain several epitopes recognized by different antibodies. Advantageously, the device of the invention allows detecting the presence of one or more microorganisms in the air, thanks to an immuno-colorimetric reaction that takes place on the support contained inside the device of the invention, at the moment in which a microorganism is present in the air entering the device. This can occur because, on the support of the device of the invention, antibodies are adsorbed that are capable of recognizing an antigenic portion of the microorganism intended to be detected.

The device of the invention is constituted by a body made of plastic or other inert material. The inert materials are a broad category of coarse particulate granular minerals used in construction and may be natural, artificial or recycled from materials previously used in construction.

On the surface of said body there is a hole from which the air present in an environment or the water to be investigated for the presence of microorganisms, enters the device.

The at least one filter present in the device of the invention is capable of capturing pathogenic and non-pathogenic contaminants (possibly present in the air or water sample) entering the device through the hole present on the body of said device. Said filter is placed inside the device. According to the invention, the filter is capable of capturing environmental contaminants, including the microorganisms possibly present in an air or water sample.

According to the invention, the filter present in the device allows said microorganisms to migrate through the support positioned inside the body of the device object of the present invention. An immuno-chromatographic process aimed at detecting the possible presence of microorganisms, in particular of the SARSCoV-2 virus, will take place on such support. Said filter is made of porous materials and the pores on the filter are between 50-100 nm, preferably between 70-90 nm in size. It was surprisingly observed that a device having a filter made of porous regenerated-cellulose material, with a pore size between 70-90 nm, allows capturing any kind of environmental contaminants. Therefore, the filter inside the device of the invention is preferably made of regenerated cellulose.

The support present in the body of the device according to the invention is thus arranged inside said device and allows displaying in real time the presence of environmental contaminants, in particular microorganisms, present in an air flow, thanks to a colorimetric reaction illustrated below. In a particularly preferred embodiment, the support in the device of the invention is a membrane, for example a membrane made of nitrocellulose, cotton fiber or glass wool.

According to a preferred embodiment of the invention (FIG. 6), the support is constituted by a first part onto which an antibody (Primary Antibody, Ab₁) conjugated with gold (Au) particles is attached, leading to the formation of the Ab₁-Au adduct. The gold particles are responsible for the formation of the color, during the execution of the test, due to the detection of the pathogenic or non-pathogenic microorganism, in particular a virus, and specifically the SARS CoV-2. The Ab₁-Au adduct is capable to recognize a portion (S_{A} epitope) of the Spike (S) protein of SARS CoV-2. A secondary antibody (Ab₂) capable of recognizing another portion (S_{B} epitope) of the Spike (S) protein of SARS CoV-2, with which it forms Ab₂:S_{B}, is attached on a second part of the support (test area). On a third part of the support (control area), the Spike (S) protein of SARS CoV-2 that will contain both S_{A} and S_{B} epitopes is attached. Finally, the support is completed with a fourth part, as a collection area of the mobile phase (buffer solution) used in excess during the execution of the test: detection phase of the pathogenic and non-pathogenic microorganism, in particular a virus and specifically SARS CoV-2.

In this case, the buffer solution is added to the hole containing the filter (which will possibly contain the pathogenic and non-pathogenic microorganism, in particular a virus and specifically SARS CoV-2). Such solution possibly containing SARS CoV-2 migrates by capillarity along the support and towards the aforementioned fourth part, initially meeting the aforesaid first part, where it finds the Ab₁-Au adduct, forming with it the Ab₁-Au:S_{A} immuno-complex which in turn migrates towards the aforesaid second part (test area) of the support, where it finds Ab₂ with which it binds to form colored Ab₁-Au:S_{A}/Ab₂:Ss. The buffer solution containing Ab₁-Au will continue its chromatographic run until it reaches the aforesaid third part (control area) of the support, where it will meet the Spike (S) protein of the virus that contains S_{A} and S_{B} and with which it will form colored Ab₁-Au:S_{A}. This control area will always stain, due to the presence of the protein (S), and thus will stain regardless of the presence of SARS CoV-2 on the filter. The aforementioned fourth part of the support absorbs the excess mobile phase.

In a particularly preferred embodiment, the device of the invention comprises a body on the surface of which there is a single hole. In said particularly preferred embodiment, the device of the invention comprises a single filter and a single support, said support being preferably a membrane, even more preferably a nitrocellulose membrane. The support (or membrane) present in the body of said device is provided with a first part comprising an examination portion (or test area) and with a second part comprising a control portion (or control area).

In the present invention, the examination portion may also be referred to as the “test area”.

In the present invention, the control portion may also be referred to as “control area”. According to the invention, the first part of the membrane comprises at least one primary, highly specific antibody that selectively recognizes an epitope of the microorganism to be detected in the air or water, and a secondary antibody. Such at least one primary antibody is associated with a secondary antibody conjugated to an enzyme, usually horseradish peroxidase or alkaline phosphatase. These two enzymes are used to amplify the signal in the photometric assays because they catalyze the conversion of the chromogenic enzyme substrate, such as 3,3′,5,5′-tetramethylbenzidine (TMB) and/or 2,2′-azino-di-[3-ethyl benzoyl thiazolinone-6-sulfonic acid], for the detection of the analyte, which in this case is the antigen. The secondary antibody is capable to recognize and bind the primary antibody present in the first part of the membrane and/or in the examining portion of said membrane.

The at least one primary antibody that selectively recognizes an epitope of the microorganism to be detected in the air or water and the enzyme-conjugated secondary antibody may be present on the first part of the membrane and/or on the examination portion of the first part of the membrane.

According to the invention, the first part of the membrane and/or the examination portion of said first part of the membrane may comprise a primary antibody or a plurality of primary antibodies. In particular, said primary antibody or said plurality of primary antibodies are attached on the membrane according to known techniques. According to the invention, at least one primary antibody and one secondary antibody conjugated with an enzyme selected from peroxidase or alkaline phosphatase necessary for the colorimetric detection activity are deposited in the first part of the support. In other words, said at least one primary antibody and at least one secondary antibody conjugated with an enzyme selected from peroxidase or alkaline phosphatase and a corresponding substrate may be present in the first part of the membrane, but not at the examination portion. In an embodiment, at least one primary antibody and at least one secondary antibody conjugated with an enzyme selected from peroxidase or alkaline phosphatase and a corresponding substrate may be present only in the examination portion of the first part of the membrane.

A standard protein specific to the microorganism to be detected is attached in the control portion of the membrane of the device of the invention, said standard protein being identical to that recognized by a primary antibody. In particular, in the presence of the microorganism in the air or water sample to be investigated for the presence of a specific microorganism, a conjugate complex of the primary/secondary antibody with the microorganism will be formed in the examination portion present in the first part of the membrane and then a standard protein/primary antibody/secondary antibody complex will be formed in the control portion of the membrane.

In a particularly preferred embodiment, said microorganism intended to be detected by the device of the invention is SARS-CoV-2.

Therefore, in a preferred embodiment, the standard protein present in the control portion, said standard protein being identical to that recognized by the primary antibody and specific to the microorganism to be detected, is a protein of SARS-CoV-2. In a particularly preferred embodiment, said standard protein is the Spike protein of SARS-CoV-2. Said Spike protein of SARS-CoV-2 is attached in the control portion of the membrane of the device of the invention according to known techniques.

SARS-CoV-2 has four different proteins: S protein (spike), E protein (envelope), M protein (membrane), and N protein (nucleocapsid). The N protein contains the RNA genome while the S, E, and M proteins together create the viral envelope. The Spike protein (S protein) allows the virus to attach to the membrane of a host cell. The Spike protein (S protein) contains two subunits, S1 and S2. S1 contains a receptor binding domain (RBD), which is responsible for recognition of and binding to the cell surface receptor. The S2 subunit is the “stem” of the structure, which contains other basic elements required for the membrane fusion. The Spike protein is the common target of antibodies and vaccines.

In a preferred embodiment, the first part of the membrane present in the device of the invention comprises at least one primary antibody capable of recognizing and binding an antigenic portion of a protein of the SARS-CoV-2 virus.

In a particularly preferred embodiment, the device according to the invention has at least one Spike-RBD anti-SARS-CoV-2 primary antibody arranged in the first part of the membrane and/or in the examination portion of the first part of the membrane. The Spike-RBD anti-SARS-CoV-2 antibodies recognize and bind, on the S1 protein of the SARS-CoV-2 virus, the binding domain of said Spike S1 protein to the receptor (RBD) of the infected host. Said at least one Spike-RBD anti-SARS-CoV-2 primary antibody is preferably produced in mouse or rabbit.

In a preferred embodiment, the at least one secondary antibody is present on the first part of the membrane and is specific for the primary antibody. Said secondary antibody may be a mouse or rabbit antibody produced in goat or sheep. The secondary antibody is produced against the host species used to generate the primary antibody. For example, if a primary antibody produced in rabbit is used, an anti-rabbit secondary antibody produced in a host species other than rabbit, e.g., a goat, will need to be used. In a particularly preferred embodiment of the device of the invention, the test area of the membrane in the device has at least one Spike-RBD anti-SARS-CoV-2 antibody attached on its surface and the control area of the membrane of said device has at least one antigen attached on its surface, in this case the Spike protein.

According to the invention, the addition of a defined volume of buffer solution for chromatographic development into the hole of the device of the invention will lead the microorganism or virus (possibly present in the air or water) through the membrane, preferably made of nitrocellulose, by adsorption through chromatography, towards the examination portion and then the control portion. In the first part of the membrane, the buffer solution that is inserted through the hole of the device will re-suspend the primary antibody and the secondary antibody by leading them towards the examination portion and the control portion, whereby the conjugate complex of the primary/secondary antibody/microorganism will attach in the examination portion while the conjugate complex of the free primary/secondary antibody will attach on the control portion with the standard protein.

Thus, the device of the invention operates through a particular type of chromatography, which is the immuno-chromatography. The chromatography is a separation technique of the components of a mixture, based on the distribution of its components between two phases, one stationary and one mobile moving along a defined direction. In the case of the present invention, due to the presence of a microorganism for which antibodies are present on the membrane or support present inside the device, an immuno-complex is formed.

The laminar flow immuno-chromatography is an investigation based on the antigen-antibody reaction on capillary beds formed by polymer strips with capabilities of capillary fluid transport. The sample, possibly including a substance of interest, once placed on said membrane, will migrate by capillarity on said polymer strips.

According to the invention, in case of the presence of microorganisms of interest, for example a virus, both parts (examination portion and control portion) present on the support of the device of the invention will exhibit a certain staining. Said staining will be detected by automated UV/VIS reader or directly by visual observation. The concentration of the microorganism is attained by interpolation between the signal intensity achieved by the reader and a standard scale made at different concentrations of the standard protein.

Advantageously, the device of the invention is particularly effective not only in detecting the presence of microorganisms in the air or water, for example viruses, but also in quantifying their concentration. In fact, thanks to the reaction between the primary antibodies present in the examination portion of the membrane and thanks to the further control carried out in the control portion of the membrane, the device of the invention allows to quickly detect the presence of a certain microorganism in a certain environment, for example in an environment with forced circulation of air. This is even more advantageous in case of detection of particularly infectious and aerosol-transmitted microorganisms, such as for example the SARS-CoV-2 virus. As a result, the device object of the present invention is certainly useful in a pandemic emergency situation, such as the present one.

Furthermore, even more advantageously, the device of the invention allows the detection of all the microorganisms, pathogenic and non-pathogenic, including viruses, for which antibodies, particularly primary antibodies are available. Therefore, the present device is not limited to the detection of the SARS-CoV-2 virus but can also be used for the detection of other microorganisms of interest, to which primary antibodies are available.

Advantageously, in order to increase the analytical specificity of the test relating to the identification of a microorganism, several devices according to the invention may be arranged in series, on which antibodies directed against different marker antigens of the same microorganism are adsorbed.

Even more advantageously, in order to multiply the analytical power of said test, several devices according to the invention can be arranged in series, on which antibodies directed against different marker antigens of different microorganisms are adsorbed.

Thus, the device of the invention is capable of sampling the circulating/recirculating air of rooms with forced circulation of air and detecting the possible presence of the contamination, for example by viruses.

Therefore, a process for detecting the presence of one or more environmental contaminants in an environment through the device of the invention provides for obtaining an air sample that will enter the device of the invention through the hole present on the body of said device. The air flow entering the device according to the invention will undergo the immuno-chromatographic reaction on the support (or membrane) of said device, first on the examination portion and then on the control portion of said support (or membrane). The last step of the process (optional) is to evaluate the concentration of contaminant present in the air sample by relating it to the standard concentration scale.

The device of the invention also proved to be effective in determining the presence of contamination, for example by viruses, even in a water sample.

Further object of the invention is a system comprising the device heretofore described and an air circulation/recirculation plant, said device being installed in said air circulation/recirculation plant.

In the system of the invention, the device of the invention is arranged so that the filter of said device is positioned in the frontal direction with respect to the air flow of the part of the air circulation/recirculation plant in which there is the return duct, in case of recirculation, or expulsion section in case of circulation, and from which a known sample of air/minute passes.

In other words, the filter of the device of the invention appears to be located in front of the air flow passing through the return ducts of said air recirculation/circulation plant. In a particularly preferred embodiment, the filter of the device of the invention is located inside the air return duct or air exhaust length in the case where the air is completely exhausted and not partially recycled (FIG. 5). In a particularly preferred embodiment, said air circulation/recirculation plant is an air conditioning plant. In this particularly preferred embodiment, the filter of the device according to the invention is arranged in front of the part of the air circulation/recirculation plant in which there is the return duct or exhaust section and through which a known sample of air/minute passes. Said known volume of air/minute is useful for quantifying the microorganism load, for example the viral load, in the environment. The system of the invention is particularly advantageous in detecting the presence of microorganisms, for example pathogenic microorganisms, such as viruses, which may be possibly present in environments with forced circulation of air, such as for example in the hospitals, offices, public or private transport means, restaurants and airports, shopping malls, gyms, shops, and more generally indoor work environments. Furthermore, said system also allows detecting the concentration of the microorganisms, for example pathogenic microorganisms such as the viruses, that may be possibly present in said environments. This is even more advantageous in case of detection of particularly infectious and aerosol-transmitted microorganisms, such as for example the SARS-CoV-2 virus. As a result, the system described in the present invention is certainly useful in a pandemic emergency situation.

### BRIEF DESCRIPTION OF THE FIGURES

In FIG. 1 the device according to the invention is depicted.

In FIG. 2 the system according to the invention during the air sampling step is depicted.

In FIG. 3 the mechanical operation of the system according to the invention is depicted.

In FIG. 4 the system of the invention is depicted.

FIG. 5 shows the arrangement of the device of the invention inside an air conditioning plant for rooms.

In FIG. 6 the device according to an embodiment of the invention is further depicted.

### DESCRIPTION OF THE FIGURES

In FIG. 1 the device 100 comprising a body 10, a hole 20, a filter 21 and a membrane (or support) 30 is depicted. Said hole 20 is arranged on the surface of the body 10 of said device 100. The filter 21 and the membrane 30 are arranged inside the device 100. The membrane 30 is comprised of a first part 31 and a second part 32. On the first part 31 of the membrane 30 there is an examination portion 41, whereas on the second part 32 of said membrane 30 there is a control portion 42. On the surface of the examination portion 41 in the first part 31 of the membrane 30, it is adsorbed at least one primary antibody (not shown in Figure) bound by a secondary antibody conjugated to an enzyme selected from peroxidase or alkaline phosphatase, and a corresponding substrate. Said primary antibody and secondary antibody conjugated with an enzyme selected from peroxidase or alkaline phosphatase and a corresponding substrate also may be present in the first part 31 of the membrane 30, but not at the examination portion 41. They are not shown in FIG. 1.

The control surface 42 in the second part of the membrane 30 comprises a standard protein specific to the microorganism to be detected and not shown in FIG. 1.

In FIG. 2 the system according to the invention during the air sampling step is depicted. Below the indication of the elements involved in the operation of the device according to the invention in the system is set forth:
1. button
2. piston
3. cartridge
4. switch
5. optical sensor/warning light.

In FIG. 3 the mechanical operation of the system according to the invention is depicted. Below the indication of the elements involved in the operation of the device, according to the invention, comprised in the system is set forth:
1. button
2. piston
3. cartridge
4. switch
5. optical sensor/warning light.

In FIG. 4 the system 300 comprising the device 100 and an air circulation/recirculation plant 200 is depicted. The device 100 is installed in said air circulation/recirculation plant 200. Said device 100 comprises a body 10, a hole 20, a filter 21 and a membrane (or support) 30. The filter 21 and the membrane 30 are arranged inside the device 100. Said hole 20 is arranged on the surface of the body 10 of said device 100. The membrane 30 is comprised of a first part 31 and a second part 32. On the first part 31 of the membrane 30 there is an examination portion 41, whereas on the second part 32 of said membrane 30 there is a control portion 42. At least one primary antibody and one secondary antibody conjugated to peroxidase or alkaline phosphatase, and a corresponding substrate (which are not shown in FIG. 4) are adsorbed on the first part of the membrane 31. At least one primary antibody (not shown in FIG. 4) is attached to the surface of the examination portion 41 in the first part 31 of the membrane 30.

On the control surface 42 in the second part of the membrane 30, a standard protein specific to the microorganism to be detected (not shown in FIG. 4) is attached.

The filter 21 of said device 100 is arranged in a frontal direction with respect to the air flow passing through the return/exhaust ducts of said air recirculation/circulation plant. The arrow in FIG. 4 depicts the direction of the air flow.

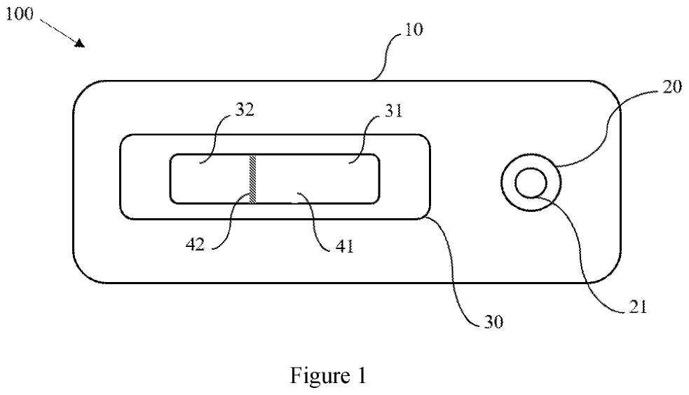

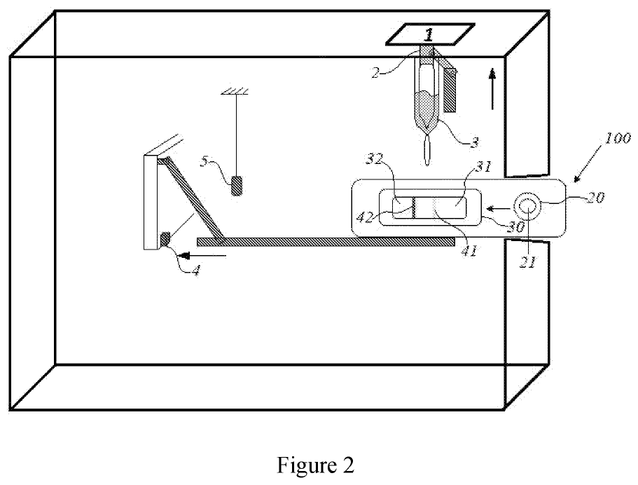

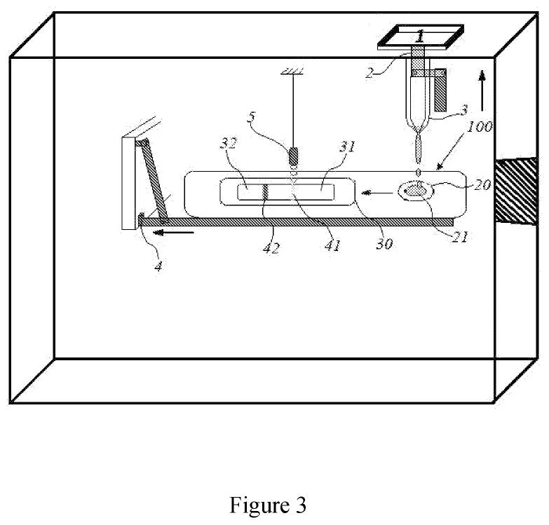

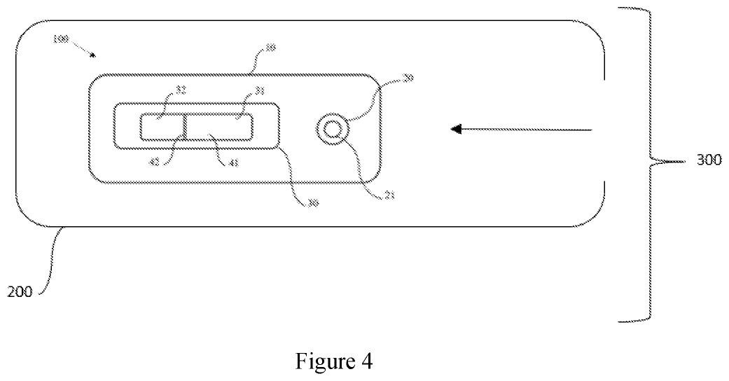

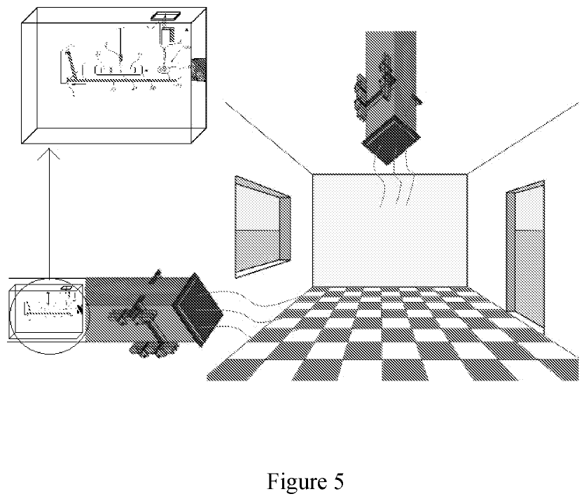

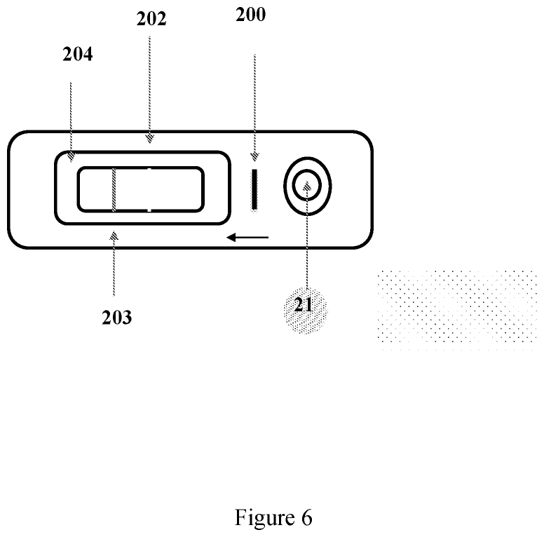

FIG. 5 shows the arrangement of the device 100 and the air recirculation/circulation plant 200 in a room. In particular, the device 100 is installed inside the air circulation/recirculation plant 200.

In FIG. 5 two ducts through which an air flow passes (top and left of FIG. 5) are depicted. Inside each of the air ducts there are two arrows indicating the direction of the air flow entering and leaving the environment. In particular, the air duct in the top center of FIG. 5, located on top of the room, is a supply duct. The air is fed into the room through said supply duct and drawn out of the room through the return duct depicted on the lower part of the left wall in FIG. 5. Arranged frontally with respect to the return duct there is the device of the invention, which is circled in FIG. 5 and depicted in larger size in the upper part of said FIG. 5.

In particular, it is the filter of the device of the invention that is placed in frontal direction with respect to the air flow drawn from the environment by the return duct. In FIG. 6 a further embodiment of the device according to the present invention is depicted, according to which there is a filter 21, a first part 200 of the device, which corresponds to the Ab₁-Au adduct, a second part 202 of the device, which corresponds to the test area on which the Abe secondary antibody is attached, a third part 203 of the device, which corresponds to the control area, on which the Spike (S) protein of SARS CoV-2 is attached, and a fourth part 204 which corresponds to the collection area of the excess mobile phase.

### EXPERIMENTAL SECTION

### Example 1

Implementation of the Device of the Invention Capable of Detecting SARS-CoV-2

A device capable of detecting the presence of the SARS-CoV-2 virus in air or water samples was made by using a nitrocellulose membrane from the Merck-Millipore company as a support for said device. On one part of the nitrocellulose membrane, i.e., on the examination portion of the first part of said membrane, anti-SARS-CoV-2 Spike-RBD antibodies were attached that recognize and bind, on the S1 protein of the SARS-CoV-2 virus, the binding domain of said Spike S1 protein to the receptor (RBD) of the infected host. A conjugate complex of the primary/secondary antibody was adsorbed on the first part of the membrane. In other words, the primary/secondary antibody complex that was used to perform the present experiment was made by a Spike-RBD anti-SARS-CoV-2 primary antibody and a secondary antibody binding said Spike-RBD anti-SARS-CoV-2 primary antibody. Said secondary antibody was a mouse antibody produced in goats.

Said secondary antibody was conjugated to peroxidase. The substrate of said peroxidase, the compound 3,3′,5,5′-tetramethylbenzidine, was also present in the examination portion of the first part of the membrane. In the control portion in the second part of the membrane, the standard Spike S1 protein of SARS-CoV-2 was attached.

Once the device of the invention was assembled, tests were performed to verify its operation. All required safety and security procedures were followed in order to perform these tests.

It was observed that the addition of 500 μl of buffer solution for the chromatographic development, constituted by Phosphate Buffer pH 7.4, in the hole of the device of the invention led the air flow through the membrane by adsorption through chromatography, towards the examination and control part. The microorganism or virus, in the present case SARS-CoV-2 possibly present in an air sample, first interacts with the conjugate complex of the primary/secondary antibody and binds itself in the test portion. At this point, the conjugate complex of primary antibody/secondary antibody continues the chromatographic migration and binds itself in the control portion of the second part of the membrane.

From the ratio of the optical signal intensity on the test portion to the optical signal intensity on the control portion, the concentration of SARS-CoV-2 in an air sample can be calculated.

### Example 2

Mechanical Operation of the Device According to the Invention

The mechanical operation of the device according to the invention is depicted in FIG. 3. In sequence:
the Phosphate Buffer pH 7.4 for the chromatographic development is pushed into the transparent envelope. This is accomplished by means of a carriage to which the swab is constrained and on which it moves/translates/slides and locks at the end of the travel.
the operator pushes the button (depicted in FIG. 3 by the number 1), the first cartridge is punctured and the Phosphate Buffer solution (pH 7.4) falls on the device of the invention at the hole containing the filter on which the virus (or other microorganism to be detected) possibly present in the sampled air that has passed through the air conditioning return system has been adsorbed. This is accomplished by means of a plunger (depicted in FIG. 3 by the number 2) directly placed below the button that squeezes the cartridge.

At this point, the operator can see in real time if the reaction is taking place and therefore the appearance of the color on the control portion (depicted in FIG. 3 by the number 42) and possibly on the examination portion (depicted in FIG. 3 by the number 41), if in the air sampled that has passed through the return of the plant there is the virus or other microorganism to be detected.

### Example 3

Electronic Operation of the Device According to the Invention

The device of the invention operates by means of a logic board programmed with the movement sequence of the parts. The sequence starts from the electric actuator of the carriage on which the Phosphate Buffer solution pH 7.4, i.e., the chromatographic development swab, is placed, returning it inside the device. At the end of the travel there is a switch that is automatically pressed by the arm, thanks to the geometry of the support on which the swab is located, which activates the actuator of the motorized carriage of the cartridge that moves the respective piston (depicted by number 2 in FIG. 3). At this point, if the swab is positive and color develops on the control portion and the examination portion of the membrane in the device of the invention, an optical sensor/warning light positioned exactly above the point where the positivity symbol should appear (i.e., above the control portion and the examination portion of the membrane) will send an alarm signal (sound, light) to the terminal to which the device is connected. The optical sensor must be synchronized to a conventional “on-off” signal when a body passing through it interrupts the signal (in this case the positivity symbol).

1. A device (100) comprising a body (10), at least one hole (20), at least one filter (21) and at least one support (30), said at least one support (30) being equipped with a first part (31) equipped with at least one examination portion (41) and with a second part (32) equipped with at least one control portion (42).

2. The device (100) according to claim 1, wherein said first part (31), has at least one primary antibody deposited on its surface, at least one secondary antibody linked to said primary antibody, said secondary antibody being conjugated with an enzyme selected from peroxidase or alkaline phosphatase, and corresponding substrate of said peroxidase and said alkaline phosphatase.

3. The device (100) according to claim 1, wherein said control portion (42) comprises at least one specific protein of at least one microorganism to be detected, wherein said at least one protein is a protein recognized by said primary antibody present on the first part (31) of the membrane (30) and/or in the examination portion (41) of said membrane (30).

4. The device (100) according to claim 1, wherein said examination portion (41) comprises at least one primary antibody recognizing the SARS-CoV-2 coronavirus.

5. The device (100) according to claim 1, wherein said examination portion (41) comprises at least one Spike-RBD anti-SARS-CoV-2 antibody.

6. The device (100) according to claim 1, wherein said support (30) is a nitrocellulose, cotton fiber or glass wool membrane.

7. A system (300) comprising the device (100) according to claim 1 and an air circulation/recirculation plant (200), wherein said device (100) is installed into said air circulation/recirculation/exhaust plant (200).

8. The system (300) according to claim 7, wherein the at least one filter (21) of said device (100) is arranged in a frontal direction with respect to said air circulation/recirculation plant (200).

9. A method of detecting at least one environmental contaminant in air or in water with the device (100) according to claim 1 or with a system (300) comprising the device (100) according to claim 1, said method comprising
obtaining an air or a water sample entering the device or the system comprising said device; wherein said air or water sample undergoes an the immuno-chromatographic reaction on the support of said device or of said system, first on the examination portion and then on the control portion of said support; thereby allowing evaluating a concentration of the at least one environmental contaminant present in the air or water sample by relating said concentration to a standard concentration scale.

10. The method according to claim 11, wherein said at least one microorganism is a pathogenic microorganism.

11. The system (300) according to claim 7, wherein said at least one filter (21) is arranged in a frontal direction with respect to the air return duct of said air circulation/recirculation plant (200).

12. The method according to claim 9, wherein said at least one contaminant is at least one microorganism.

13. The method according to claim 10, wherein said pathogenic microorganism is a virus.

14. The method according to claim 10, wherein said virus is the SARS CoV 2 coronavirus.
